(19) United States
(12) Patent Application Publication
(10) Pub. No.: US 2002/0197653 A1
(43) Pub. Date: Dec. 26, 2002

(54) System for detecting reporter gene expression

(76) Inventors: Matthew Shair, Boston, MA (US); Lawrence Chan, Cambridge, MA (US)

Correspondence Address: Choate, Hall & Stewart, Exchange Place, 53 State Street, Boston, MA, 02109, US

(21) Appl. No.: 10/091,240

(22) Filed: Mar. 5, 2002

Related U.S. Application Data
(60) Provisional application No. 60/273,736, filed on Mar. 5, 2001.

(51) Int. Cl. G01N 33/567

(52) U.S. Cl. 435/7.21

(57) ABSTRACT

An in vivo assay system for identifying chemical compounds that inhibit or promote a biological event is described. Test compounds may be assayed for their ability to induce the expression of a reporter gene which subsequently leads to the production of a reporter gene product. Preferably the reporter gene product is secreted from the cell or is membrane permeable so that the product is readily detectable. The signal from the reporter gene is preferably amplifiable so that even minute changes in expression of the reporter gene are detectable. The present invention also provided combinatorial libraries for use in the inventive assay system.

### PRIORITY INFORMATION

The present application claims priority under 35 U.S.C. § 119(e) to U.S. Provisional application No. 60/273,736, filed Mar. 5, 2001, the entire contents of which are hereby incorporated by reference.

### BACKGROUND OF THE INVENTION

Since the discovery and commercial success of drugs such as aspirin (acetylsalicylic acid) and penicillin, the search for small molecules with biological and therapeutic activity has been a major goal of medical research. One approach to finding small molecules having these activities is to laboriously screen samples of soil extracted from all parts of the world for microorganisms that secrete small molecules with a desired biological activity. Upon the identification of an activity, the next step is to isolate the desired specific strain of microbe from the multitude of microorganisms in any single soil sample, followed by the isolation of the specific molecule secreted by the microbe. This process of drug discovery is inefficient and extremely time-consuming. However, many useful therapeutic and biologically active molecules have been discovered by this method (e.g., FK506, enediyne-containing molecules).

A second approach to discovering molecules with biological and therapeutic activity is known as rational drug design. The goal of this strategy is to synthesize molecules based on a known biological structure, typically that of a protein. In most cases, the molecules are designed to bind to a specific region of the protein, such as its biologically active site or a region that interacts with another molecule or protein. An advantage of this approach is that molecules with a high degree of binding affinity and specificity can be synthesized; such molecules (e.g., protease inhibitors, finasteride/Propecia) often have potent effects on the biological process. However, one drawback of this method is that the molecular structure of biological targets must be known in great detail and with a high degree of accuracy.

Another recent approach to drug discovery is known as “functional genomics”. This strategy involves the determination of protein function on a genome-wide basis, and has become a major research endeavor accelerated by the successful sequencing of several genomes. The branch of functional genomics known as “chemical genetics” uses small organic molecules to interrogate protein function. In a manner somewhat analogous to genetic mutations, small molecule inhibitors of protein function can act as conditional modulators of the cellular process in which the proteins are involved. Analysis of the chemicals and their activities thus simultaneously reveals functional information about the protein or pathway being studied and identifies potential drug candidates that could be used to modulate that protein or pathway. Chemical genetics also provides the experimental advantage (over traditional genetics) that the chemical reagents used to probe protein function can be added to the biological system at any desired time in the assay, so that temporal and/or conditional effects may readily be studied.

For the chemical genetic approach to be maximally successful, methods for efficiently identifying small molecules of interest on a genome-wide basis must be developed. Accomplishing this goal requires both the ready availability of large numbers of chemical compounds to be screened and the availability of systems for screening them. Recent important advances in synthetic chemistry have dramatically increased the availability of small molecule compounds; some syntheses can now generate as many as a million or more compounds in a single library. There remains a need, however, for the development of useful strategies for screening such libraries quickly and efficiently.

### SUMMARY OF THE INVENTION

The present invention provides a system for detecting expression of a reporter gene in the vicinity of a solid support. In particular, the invention provides a support-bound detecting agent that undergoes a detectable change when placed in the physical vicinity of a reporter gene product. In certain preferred embodiments of the system, the reporter gene comprises a nitric oxide (NO) synthase gene and the detecting agent detects nitric oxide (NO), a reporter gene product.

The inventive system may be employed to detect reporter gene expression in any of a variety of contexts. For example, the reporter gene may be expressed in vivo or in vitro. In certain preferred embodiments, reporter gene expression occurs in vivo. Also, reporter gene expression may be used as a proxy for the detection of another biological event that ultimately results in reporter gene expression. For example, in certain preferred embodiments of the invention, the reporter gene is placed under the control of two or more interacting regulatory biomolecules. Preferably, at least one of the regulatory biomolecules is a protein. Reporter gene expression occurs only when proper interaction between or among the regulatory biomolecules is achieved. In such an arrangement, the inventive system may be employed to identify or characterize test compounds that promote or inhibit interaction of the regulatory biomolecules. For example, the regulatory biomolecules may be proteins within a signal transduction pathway linked to expression of the reporter gene.

In preferred embodiments of the invention, reporter gene expression is monitored in a high-throughput format. The inventive system therefore allows analysis of large numbers of compounds that may alter or affect expression of the reporter gene. In certain preferred embodiments, the collection of compounds assayed represents at least a portion of a combinatorial library. Preferably, the library is preferably attached to a solid support. The solid support may optionally include one or more tags representing various structural aspects of and/or synthetic steps used to create the particular attached library member. The solid support may also contain a detecting agent capable of sensing the reporter gene product. The inventive assays may be performed in high-density plates with greater than 500 wells, more preferably greater than 1000 wells, and most preferably greater than 5000 wells. The inventive assay system may also take advantage of other technological advances in high-throughput screening, including robotic machines, microarrayers and other arraying devices, high-density plates, fluorescence-activated bead sorting (FABS), CCD cameras, microscopes, fluorescence microscopy, and computer analysis.

### Definitions

Amino acid, as is known in the art, refers to an organic acid in which one of the CH hydrogen atoms has been replaced by NH₂. Preferably, an amino acid is an α-amino acid, having a formula R—CHNH₂—COOH. As is also known, there are twenty different amino acids that occur in nature and are used naturally to build proteins. Other, non-natural amino acids have also been prepared (see, for example, http://www.cco.caltech.edu/˜dadgrp/Unnatstruct.gif, which displays structures of non-natural amino acids that have been successfully incorporated into functional ion channels). Also, one or more of the amino acids may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc.

Associated with refers to a physical, spatial, or other association between two entities, e.g., a detecting agent and a solid support. Any means of association may be utilized so long as the detecting agent can act as an identifier of a particular solid support. In most embodiments of the present invention, it is preferred that the detecting agent be physically linked, directly or indirectly (e.g., by non-covalent interaction with a compound that is covalently linked to the solid support), to the solid support, preferably via a covalent linkage.

Combinatorial library is a collection of compounds whose chemical structures are related to one another in that all members of the library were or could have been produced through an iterative synthesis procedure in which a predetermined number of core molecules is passed sequentially through a collection of different reaction protocols in a manner that allows the production of a variety of different compounds by alternative functionalization or modification of the core molecules. Preferred modes of combinatorial synthesis include parallel synthesis and split-and-pool synthesis. As is known in the art, combinatorial synthesis can be performed in liquid or on the solid phase. In certain preferred embodiments of the invention, solid phase synthesis is utilized. Those of ordinary skill in the art will appreciate that the present combinatorial library definition encompasses collections of compounds whether they in fact were synthesized using a combinatorial approach as described herein or were provided (whether through synthesis, extraction, purification, or other method) separately and then combined.

Construct refers to any polynucleotide that has been manipulated by the hand of man. For example, a polynucleotide may be considered a “construct” in accordance with the present invention if it (i) is isolated from one or more compounds with which it is associated in nature; (ii) includes one or more nucleotide sequences that are separated from other sequences with which they are associated in nature; and/or (iii) is produced using recombinant techniques, such as the polymerase chain reaction (PCR), restriction endonuclease digestion, enzymatic ligation, etc. (see, for example, Molecular Cloning: A Laboratory Manual, 2nd Ed., ed. by Sambrook, Fritsch, and Maniatis (Cold Spring Harbor Laboratory Press: 1989); Methods in Enzymology (Academic Press, Inc., N.Y.); Ausubel et al. Current Protocols in Molecular Biology (John Wiley & Sons, Inc., New York, 1999); each of which is incorporated herein by reference). Preferably, the polynucleotide contains various elements that are operably linked to one another, preferably including one or more elements sufficient to allow introduction into and/or replication in a cell. For example, the construct may contain a promoter operably linked to a coding sequence, and the construct may be introduced into a cell to cause the cell to produce the encoded protein.

Fusion protein refers to a protein comprising two or more polypeptides that, although typically unjoined in their native state, are joined by their respective amino and carboxyl termini through a peptide linkage to form a single continuous polypeptide. The two or more polypeptide components can be either directly joined or indirectly joined through a peptide linker/spacer. The fusion protein may be translated by a ribosome from mRNA as a single polypeptide, or the polypeptides may be joined using synthetic or enzymatic chemistry.

Gene expression, as is known in the art and will be clear from context, refers to any or all of the steps involved in synthesizing an RNA or protein encoded by a gene. In particular, gene expression refers to one or more of the processes of transcription, splicing, capping, polyadenylation, RNA editing, translation, and post-translational modification (e.g., truncation, addition, glycosylation, phosphorylation, proteolysis, etc.).

Nucleic acid, polynucleotide, or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′fluororibose, ribose, 2′-deoxyribose, 2′-O-methylcytidine, arabinose, and hexose), or modified phosphate groups (e.g., phosphorothioates and 5′-N-phosphoramidite linkages).

Operably linked is used to describe to two segments of polynucleotide sequence that can affect each other. In a particularly preferred embodiment, one of the two segments is a sequence that binds a protein (e.g., polymerase, enhancer-binding factor, and transcription factor), and the binding of the protein to the sequence leads to the transcription of a gene sequence located in the second segment. In another particularly preferred embodiment, the binding of a molecule (e.g., nucleic acid, small molecule, protein, and peptide) to one segment may inhibit or enhance the binding of another molecule (e.g., nucleic acid, small molecule, protein, and peptide) to the second segment. For example, the first segment may comprise an enhancer, and the second may comprise a promoter whose occupancy by RNA polymerase is affected by the occupancy of the enhancer. Preferably, two operably linked segments are covalently linked, but any type of association sufficient to achieve the desired results is considered to be operably linked in the context of the present invention.

Protein or peptide is an amino acid polymer that is at least four amino acids in length. As will be clear from context, the term protein is used herein to refer both to complete proteins (i.e., a functional unit that is expressed and is active in nature) and/or to protein fragments and polypeptides. An amino acid polymer need not ever exist in nature to be considered a protein herein. In particular, the term encompasses fusion proteins comprised of two or more amino acid sequences that are found in nature but are not usually linked together in a single polymer, as well as wholly artificial sequences. Also, the term may encompass polymers that include one or more modified amino acids and/or one or more non-natural amino acids.

Regulatory biomolecule, according to the present invention, is a factor or compound that regulates expression of a gene through interaction with a partner biomolecule. A regulatory biomolecule may be a protein, a nucleic acid, a natural product, a small molecule, a complex of proteins and/or small molecules, or any other chemical agent. In preferred embodiments of the invention, at least one regulatory biomolecule is a protein or complex of proteins. More preferably, both (or all) regulatory biomolecules are proteins or protein complexes.

Reporter gene, for the purposes of the present invention, is any nucleic acid sequence that produces a reporter gene product detectable as described herein. It will be appreciated that a reporter gene need not include an open reading frame; often, however, the reporter gene will contain at least one open reading frame, preferably encoding a complete protein or functional domain thereof. In certain preferred embodiments of the invention, the encoded protein or functional domain has catalytic activity and reporter gene expression can be detected through detection of a product of catalysis.

Reporter gene product can refer to a nucleic acid gene product (e.g., a primary transcript, any spliced transcript, and/or a mRNA), a polypeptide or protein gene product, or a more downstream product that itself is only produced as a result of production of a nucleic acid or protein gene product. For example, a spliced intron could be a gene product, as could a chemical compound that is synthesized by a protein gene product. In general, a reporter gene product is any entity whose existence or form reveals that gene expression has occurred. In certain preferred embodiments of the invention, the reporter gene product is a protein gene product. In other preferred embodiments, the reporter gene product is a chemical compound that is produced by action of a protein gene product. Preferably, the reporter gene product is easily detectable using standard techniques in the art. In a particularly preferred embodiment, the reporter gene product is detectable using molecular sensors, chemical sensors, chemosensors, or biosensors currently available.

Small molecule, as used herein, refers to a non-peptidic, non-oligomeric organic all compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J Am. Chem. Soc. 120:8565, 1998; incorporated herein by reference) and pending application number 08/951,930 “Synthesis of Combinatorial Libraries of Compounds Reminiscent of Natural Products”, the entire contents of which are incorporated herein by reference. In certain other preferred embodiments, natural-product-like small molecules are utilized.

### BRIEF DESCRIPTION OF THE DRAWING

FIG. 1 shows the general scheme of the inventive assay. A library of test compounds 200 is provided attached to a solid support 100 which is also attached to a molecular sensor 300. Test compounds are identified which lead to expression of the reporter gene 400 and the resulting production of a detectable reporter gene product 500.

FIG. 2 depicts a scheme for the rapid automated screening of small molecules libraries using nitric oxide synthase as the reporter gene and nitric oxide as the reporter gene product. Fluorescence activated bead sorting can be used to rapidly sort the beads to identify those containing hits.

FIG. 3 depicts an application of the NO high-throughput assay methodology to the discovery of test compounds which disrupt protein-protein interactions. This application uses an isoform of NO synthase which requires calmodulin/Ca⁺² for activation, and PIN, protein inhibitor of NO synthase.

FIG. 4 shows the use of the NO-based reporter gene system in detecting small molecule initiated transcription.

FIG. 5 shows the use of macrophages as a system for the NO synthase reporter gene.

FIG. 6 shows a high-throughput assay design based on screening for small molecules which disrupt the Rb-E2F/DP1 complex.

FIG. 7 shows the test compound (ligand) attached to the solid support (bead) through a photocleavable linkage. The NO sensor 2,3-diaminonapthalene (DAN) is also attached to the solid support.

FIG. 8 shows the chemical structure of another NO sensor.

FIG. 9 shows a scheme for attaching the NO sensor to the solid support using a carbene insertion reaction.

FIG. 10 shows the use of a disulfide linker instead of a photocleavable linker. Test compounds can be released from the linker by the addition of cysteine.

FIG. 11 depicts the use of hydrazine to create a chemical handle (a hydrazide) that allows for linking the sensor to the solid support via a standard amine coupling reaction.

### DETAILED DESCRIPTION OF CERTAIN PREFERRED EMBODIMENTS

As discussed above, the present invention provides a system for detecting reporter gene expression. What follows is a description of certain preferred embodiments of the invention. Those of ordinary skill in the art will readily appreciate that the subsequent text is not intended to limit the scope of the invention, as defined in the claims.

In general, the inventive system utilizes a detecting agent that is associated with a solid support and responds in a detectable fashion to expression of a reporter gene in the vicinity of the solid support. In general, the detecting agent can be any compound or factor that undergoes a detectable change that correlates with a change in expression or activity of a selected reporter gene. It will be appreciated that selection of the reporter gene and the detecting agent are related to one another in that the detecting agent must be characterized by an ability to detect a change in expression of the reporter gene.

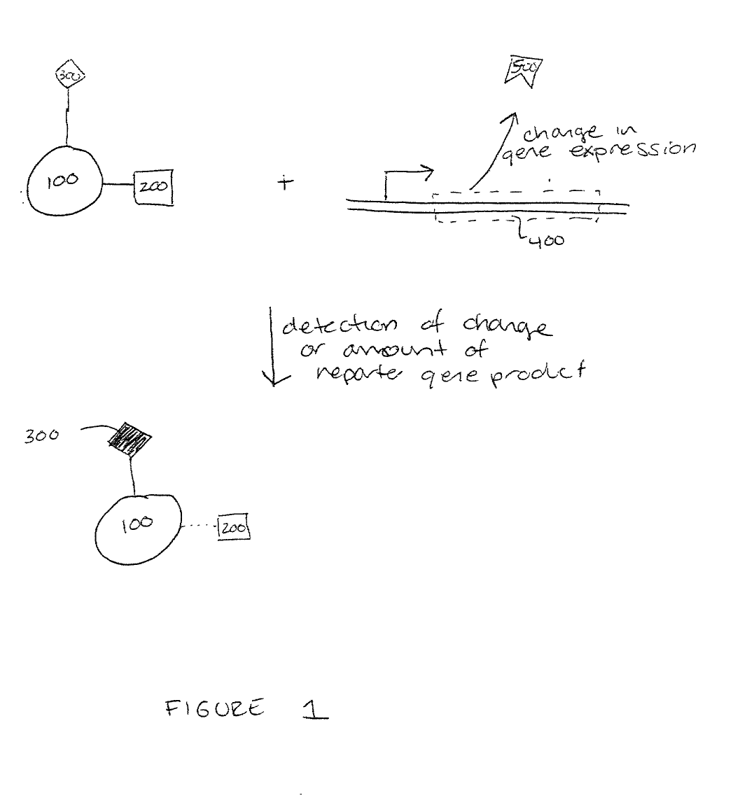

FIG. 1 presents a schematic representation of one preferred embodiment of the present inventive system. As indicated, a solid support 100 is provided that has attached a test compound 200 and a detecting agent 300. The solid support 100 is placed in the vicinity of a reporter gene 400. The test compound 200 exerts a direct or indirect effect on expression of the reporter gene 400, resulting in a change in the amount or nature of a reporter gene product 500, which change is detectable by the detecting agent 300. The shading of the detecting agent 300 is intended to represent a detectable change in that detecting agent 300. The change in the detecting agent 300 may be any detectable event, including but not limited to emission of electromagnetic radiation (e.g., fluorescence, chemiluminescence, phosphorescence), absorbance of electromagnetic radiation, change in chemical structure, radiolabeling, etc.

Test compounds 200 may be attached to the support by any available mechanism. For example, a test compound 200 may be covalently linked to the solid support 100, or may be associated with the support through a binding interaction (e.g., by means of ionic, van der Waals, electrostatic, hydrophobic, and/or hydrogen-bonding interactions). Also, test compounds 200 may be attached to another molecule that is in turn attached to the solid support 100. In certain preferred embodiments of the invention, approximately 100-1000 pmol of compound 200 are loaded onto each solid support 100.

It will be appreciated that test compound 200 may optionally be released from the solid support 100 prior to detection of its effect on reporter gene expression. A wide variety of covalent and non-covalent cleavable linkages appropriate for associating a test compound with a solid support are known in the art (see, for example, Fruchtel et al., Angew. Chem. Int. Ed. Engl. 35:17, 1996, Tables 2 and 3 of which are incorporated herein by reference). Preferably, the linkage is cleavable by exposure to acidic conditions, basic conditions, or a certain wavelength of light.

A cleavable linkage may alternatively be accomplished by linking the test compound molecules to an agent, such as a protein or polypeptide, that is sensitive to cleavage by a known enzyme or chemical cleavage agent (see, for example, glutathione-S-transferase [GST] fusion system available from Pharmacia). A wide variety of chemical (e.g., cyanogen bromide) and enzymatic (e.g., trypsin, chymotrypsin, carboxypeptidase Y, precursor protein processing enzymes, etc.) protein cleavage agents with specific recognition sites are known in the art (see, for example, Hermodson, Methods in Protein Sequencing Analysis, ed. Elzinga, Humons Press, Clifton, N.J., pp. 313-323, 1982; see also Sigma Chemical Company catalog listing of Protein Analysis Reagents; each of which is incorporated herein by reference). Alternatively, the test compound molecules may be linked to a nucleic acid molecule that contains a cleavage site for a restriction endonuclease or other nucleic acid cleaving agent (e.g., a ribozyme). Test compounds may also be attached by means of, for example, a disulfide linker that can be cleaved by exposure to reducing conditions (e.g., the interior of a cell, P-mercaptoethanol, dithiothreitol).

Severable linkage may also be accomplished if the association between the test compound and the solid support can be competed out by exposure to a competitive agent. For example, test compounds fused to GST will bind to a solid support to which glutathione is attached, and this binding can be competed by free glutathione.

Preferred severable linkages are those that allow the extent of compound release to be controlled by exposure to varying degrees of release signal, and therefore allow control of the concentration of test compound being assayed. For example, the extent of severance of photo-cleavable linkages can generally be varied by altering the time of exposure to radiation of the appropriate wavelength. Similarly, the extent of severance of competable attachments can be adjusted by altering the concentration of competitive agent.

Also, in many embodiments of the invention, it is preferred that the association of the detecting agent 300 with the solid support 100 be stable under the conditions of the assay. and also under conditions that would result in release of the test compound 200 from the solid support 100. Preferably the detecting agent 300 is covalently linked to the solid support 100.

Solid Support

In preferred embodiments, the test compounds are provided in association with a solid support to which multiple molecules are directly or indirectly attached. Preferred support materials include solid polymeric materials such as, for example, polydextran, sephadex, polystyrene, polyethylene glycol, polyacrylamide, cellulose, agarose, polysaccharides, and combinations thereof. Glass, latex, acrylic, or ceramic supports may also be employed, as may any of a variety of encapsulation matrices The solid support may be of any shape or size; however, as would be appreciated by one of skill in this art, smaller size solid supports are generally preferred. The mean diameter of the solid supports is preferably in the range of 1 to 1000 μm, and more preferably in the range of 10-500 μm.

In a particularly preferred embodiment, the solid supports are TENTAGEL (trademark of Rapp Polymere GmbH) beads. Preferably the solid support has chemically reactive sites available for attaching the test compound and the detecting agent. The solid support should preferably contain 0.01 to 10 mmol of chemically reactive sites per gram of solid support, more preferably 0.1 to 0.5 mmol/g. Chemically reactive sites useful in the present invention include nucleophiles (e.g., hydroxyl groups, thiol groups, amino groups) and electrophiles (e.g., carbonyl-containing groups, aldehydic groups, alkyl halides, acid chlorides, amides, carboxylic acids, esters).

Preferably the solid support itself does not substantially interfere with any aspect of the assays being performed.

Detecting Agent

The detecting agents utilized in the present invention may be any agent which undergoes a detectable change in at least one of its properties or in its structure upon exposure to the reporter gene product. The detecting agent may be any chemical compound, protein, nucleic acid, organometallic compound, small molecule, peptide, etc. Examples of properties of the detecting agent which may undergo a change are fluorescence, phosphorescence, absorbance, chemiluminescence, enzymatic activity, etc. Preferably there is a change in an optical property, wherein the change can be observed spectroscopically. The detecting agent may also undergo a chemical change upon exposure to the reporter gene product. For example, the reporter gene product may be an adduct with the detecting agent. Preferably, the change in the detecting agent is easily detectable and is very sensitive to even minute quantities of the reporter gene product.

In the case wherein nitric oxide is the reporter gene product, certain preferred NO sensors include diaminofluorescein and 2,3-diaminonaphthalene (DAN).

Reporter Gene

Reporter genes useful in the present invention include any gene whose direct or indirect product is easily detectable. The gene may encode an RNA transcript (e.g., mRNA) which is detectable. The gene may also encode a protein which is detectable directly or indirectly through its enzymatic activity. The reporter gene product is preferably secreted by the cell and/or is able to easily through the cellular membrane. In certain preferred embodiments, the level of the reporter gene product is quantifiable. Preferably, detection of the reporter gene product is performed without the addition of another reagent.

A particularly preferred reporter gene is nitric oxide synthase, and enzyme which catalyzes the production of NO from arginine. The nitric oxide synthase may be any isoform, derivative, or homologue.

Test Compound

The compounds to be screened in the inventive assays may be provided by any means known in the art. The test compounds may be polynucleotides, peptides, proteins, small molecules, organic molecules, inorganic molecules, peptidomimetics, antibodies, or other chemical compounds. The compounds may be prepared by purification or isolation from a source (e.g., plant, fungus, animal, bacteria, soil sample, etc.), or by synthesis. The synthesized compounds may be created by more conventional one-by-one synthetic methods or by combinatorial chemistry methods through rapid parallel and/or automated synthesis. The compounds may be provided in crude or pure forms. The compounds may be natural products or derivatives of natural products. In another preferred embodiment, the compounds are provided from the historical compound files of large pharmaceutical and chemical companies. Preferably, the compounds are provided as libraries of chemical compounds.

System for Screening Small Molecule Libraries using Nitric Oxide Synthase

The present invention provides a method of identifying reagents capable of affecting protein-to-protein interactions by screening small molecule libraries in high-throughput for molecules with a desired protein binding activity. In an aspect of the present invention, the method utilizes nitric oxide synthase (NOS) as a reporter of gene activation in a high-throughput screen of small molecule libraries synthesized by combinatorial chemistry to identify small molecules that affect a protein-protein interaction of interest. In addition, solid supports used in the syntheses of the libraries contain standard encoding methods to record the route of syntheses, and also contain molecular sensors of nitric oxide (NO) to detect NO generated by NOS when the gene encoding NOS is transcriptionally activated.

As a method of detecting a biological event, reporters of gene activation are commonly used to identify reagents which activate gene expression. Reporter genes which convert protein binding events, as in a yeast two-hybrid experiment, to colorimetric or fluorescent readouts have been developed based upon transcriptionally controlled enzymatic reactions. The most popular systems include luciferase, β-lactamase, secreted alkaline phosphatase (SEP), and green fluorescent protein (GFP).

Nitric oxide (NO) is a diatomic molecule which has biological functions in virtually every organ in mammals (Koshland, Science 258:1861, 1992; incorporated herein by reference). The production of NO is mediated by a family of nitric oxide synthases (NOS). Genes encoding NOS have been found in mammals, chickens, insects, and invertebrates (snails). In mammals, the NOS family of proteins consists of three isoforms. Neuronal NOS (nNOS, sometimes referred to as brain NOS; bNOS) and endothelial NOS are two isoforms expressed at low levels. The most widely expressed isoform is the inducible NOS (iNOS). iNOS has been identified in a diverse set of mammalian tissues and was originally purified from a macrophage cell line. iNOS is a constitutively active enzyme and catalyzes the conversion of arginine to citrulline and NO. Currently, it is known that induction of iNOS occurs in the presence of numerous molecules, including inflammatory cytokines, bacterial lipopolysaccharides, interleukins, tumor necrosis factors, and cyclic adenosine monophosphate (cAMP). The promoter region of iNOS contains binding sites for several transcription factors such as NFκB, C/EBP, CREB/ATF.

There are several advantages to using NOS as a reporter of gene activation when compared to current conventional reporters. One advantage is the ability of NOS to generate NO without the addition of cofactors. Another advantage is the ability of NO to pass through cell membranes easily facilitating detection. An additional advantage is the ability to utilize enzymatic or catalytic reactions to amplify the signal (levels of NO) for colorimetric or fluorescent detection. Such detection methods allow automation and thus, ultrahigh throughput screening of molecules.

In a preferred embodiment of the present invention, small molecules that disrupt or inhibit a protein-protein interaction of interest can be identified. A system for detecting changes in protein-protein interactions using gene activation to detect a signal is designed such that an intact protein-protein interaction of interest represses transcription of a gene encoding a nitric oxide synthase (NOS). By way of example and not limitation to the present invention, a protein that binds to the promoter region of the iNOS gene to activate transcription is complexed with other proteins to prevent binding to the promoter or activation of transcription. In macrophages, the gene encoding iNOS is activated by the transcription factor NFκB in the presence of stimulatory cytokines such as IFN-γ and lipopolysaccharides (LPS). Normally the gene is dormant in part due to interactions of NFκB with IκB which masks the nuclear translocation domain of NFκB. In the presence of the cytokines, the NFκB-IκB interaction is disrupted, and NFκB can then translocate into the nucleus. Consequently, NFκB binds to many sites including the promoter region of the gene encoding iNOS to activate transcription of iNOS. The transcription of iNOS ultimately results in the production of NO gas. In this naturally occurring system of gene activation, libraries of small molecule can be screened for molecules that directly or indirectly disrupt the NFκB-IκB complex to free NFκB resulting in transcription of iNOS and the production of NO gas.

Small molecules can affect protein-protein interactions directly or indirectly. A small molecule directly affects protein-protein interaction by physically binding to one or more components of a protein complex. Alternatively, small molecules can indirectly affect protein-protein interactions without binding directly to a component in the complex. For example but without limitation, a small molecule can target an enzyme or a precursor involved in the synthesis and/or post-translational modification of a protein in a multiprotein complex to inhibit proper formation of that protein.

Any transcription factor with binding partners that inhibit the activation of transcription can be examined in a screen for small molecules that disrupt protein-protein interactions using NOS as a reporter of gene activation. For example without limitation, transcriptional activators such as the tumor suppressors p53 and Rb (for a review of the Rb tumor suppressor protein and its regulation of E2F, see: Weinberg Cell 81:323, 1995; incorporated herein by reference) are involved in protein complexes that mask and inhibit transcriptional activation domains. In the case of p53, an MDM2/p53 complex masks the transcriptional activational domain of p53 to inhibit transcriptional activation by p53. For Rb, an E2F/DP1/Rb complex inhibits the activation of transcription by E2F. Since methods of constructing DNA promoters containing protein binding sites are well known in the art (see, for example, Molecular Cloning: A Laboratory Manual, 2nd Ed, ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999); Transcription and Translation (B. D. Hames & S. J. Higgins eds. 1984); each of which is incorporated herein by reference), DNA binding sites for any transcriptional activator can be constructed into the promoter region of the gene encoding iNOS.

In another preferred embodiment of the present invention, libraries of small molecules are synthesized on solid phase resin beads. The small molecules can then be delivered from the bead by photolysis. Syntheses of libraries using combinatorial chemistry are well known in the art (see, e.g., “Combinatorial Chemistry”, Chem. and Eng. News 43, Feb. 24, 1997; Thompson et al. Chem. Rev. 96:555, 1996; Furka et al. Int. J. Pept. Protein Res. 37:487-493, 1991; Czarnik Curr. Op. Chem. Biol. 1:60, 1997; each of which is incorporated herein by reference). Beads are tagged by standard encoding methods to identify the molecule on the surface of the beads. The beads are also labeled with molecular sensors of NO. Preferably the molecular sensors have spectral properties that are altered in the presence of NO. Preferably the alteration in spectral properties is a shift from a non-fluorescent state to a fluorescent state. It is recognized that any molecule that enables the spectroscopic determination of the presence of nitric oxide may be utilized in the present invention. For example without limitation, a fluorescein-based molecule that is activated by the presence of NO (or a reaction product of NO) can be used as a sensor.

The combination of the small molecule, the tag, and the sensor on the same bead greatly facilitates the identification of a molecule shown to disrupt or inhibit a protein-protein interaction by production of NO. Since each bead contains a different molecule from the library for screening and a sensor of NO production, a positive signal indicating NO production and hence, disruption or inhibition of a protein-protein interaction, can easily be traced to the molecule in the library responsible. Another advantage of delivering the small molecule library on a bead with a tag and a sensor of NO is that the local concentration of the fluorescent sensor on the surface of the beads is higher than the concentration of the same fluorescent molecules free in solution. As a result, the sensitivity of detecting NO by fluorescence is greatly enhanced.

In another preferred embodiment of the present invention, the high throughput screen of the present invention can be performed in high density wells (i.e., 1536 or 6000 well format). Identification of fluorescent beads is performed visually or utilizing a CCD (charged coupled device) camera and related software known to those skilled in the art. Alternatively but without limitation, fluorescent beads can be sorted away from non-fluorescent beads by fluorescence-activated bead sorting (FABS). As previously described, identification of the active molecule on fluorescent beads is easily accomplished by decoding the tags produced by standard encoding methods.

In another preferred embodiment of the present invention, high-throughput screening of small molecule libraries using NOS as a reporter of gene activation can be used to identify small molecules that affect any protein-protein interaction of interest. In this embodiment, an artificial transcription factor is constructed composed of four components. The first component is a polypeptide comprising a DNA-binding domain. Examples of DNA-binding domains are well known in the art. DNA-binding domains that are commonly used for fusion with other proteins include lexA and GAL4. The second component fused to the first component is a protein/polypeptide of interest (X). Fusion is defined as the formation of one polypeptide from two shorter polypeptides by creating a peptide bond between the two shorter polypeptides. A third component is a second protein or polypeptide of interest (Y) known to bind to protein X. Protein Y is fused to a transcriptional activation domain. Examples of activation domains are also well-known in the art (e.g., B42). The resulting system allows any two physically interacting polypeptides to link a transcriptional activation domain to a desired DNA-binding domain. By designing a promoter to contain DNA-binding sites for the desired DNA-binding domain and integrating the resulting promoter upstream of a gene, one of ordinary skill in the art can construct a promoter to activate a gene of interest using this artificial transcription factor chimera. As a result, a small molecule that inhibits or disrupts the Protein X-Protein Y complex will repress transcription of the gene of interest.

By way of example and not limitation, expression of a protein X-LexA fusion (DNA binding domain), a protein Y-B42 fusion (activation domain), and nNOS (an isoform of NO synthase which requires calmodulin/Ca⁺² for activation) is placed under the control of a galactose inducible promoter. Galactose dependent expression allows the small molecule to be introduced into the experiment before protein X and protein Y have been synthesized or at any time subsequent to synthesis of protein X and protein Y. The DNA binding site for the DNA binding domain is placed upstream of a DNA sequence encoding PIN (protein inhibitor of NO synthase). PIN is a recently discovered protein that inhibits the activity of nNOS and is believed to be involved in nNOS regulation. Therefore, binding of protein X to protein Y brings the activation domain (AD) into close proximity to the gene encoding PIN resulting the activation, transcription, and subsequent synthesis of PIN. In the presence of PIN, nNOS is inhibited. As a test of nNOS inhibition, the addition of calcimycin, which activates calmodulin by increasing intracellular Ca⁺² levels, does not result in NO production. Therefore, the end result of protein X binding to protein Y is a bead with an unactivated NO sensor. If a small molecule disrupts the protein X-protein Y complex, then transcription of PIN is not initiated, and PIN protein is not synthesized. In the absence of PIN, nNOS is activated, and upon addition of calcimycin, NO generation leads to fluorescent beads which can be detected and separated by FABS. Any protein-protein interaction could be rapidly screened using our system with the aid of automated sorting.

In another preferred embodiment, a high-throughput screen of small molecule libraries utilizes the production of nitric oxide as a method of identifying a small molecule that enhances the protein-protein interactions between two proteins. Enhancing protein-protein interactions is defined as increasing the physical binding constant of one protein for the other protein. It may be desirable for two proteins or polypeptides which normally have a low affinity for one another to have an increase binding affinity for one another. A first protein X is constructed fused to the DNA-binding domain (DBD) of another protein. The DNA binding site corresponding to the DNA-binding domain is placed in the promoter region of the gene encoding iNOS. A second protein Y which has a low affinity for protein X is fused to an activation domain (AD) that will activate transcription of the gene encoding iNOS. In this system, when the Protein Y-AD fusion protein is brought into close proximity to the promoter of iNOS by a molecule that binds to both X and Y, the activation domain of the Protein Y-AD fusion protein will activate transcription of iNOS to generate NO.

The present invention also provides for a reporter of gene activation. There are numerous advantages to the iNOS reporter gene system over current technology. First, the system does not require the addition of external cofactors as in the luciferase and β-galactosidase reporter genes. The end product of gene expression, NO gas, is highly diffusible and, by nature, cell permeable. Therefore, cells which have been activated in the assay will secrete NO gas which will be detected chemically. iNOS is also capable of enzymatic amplification. Inducible NO synthase is a constitutively active enzyme that, once switched on, remains in the on position. As a result, an iNOS based reporter gene should be highly sensitive to low levels of transcription.

Currently in order to modify the readout of various reporter genes, it is required that the reporter gene itself be exchanged. For instance, switching between calorimetric and fluorescent readouts requires that cell-based assay systems be generated with reporter genes which produce a specific type of readout. An advantage of the iNOS reporter system is that the readout has been converted to a secreted chemical product (NO) which can interact with a number of different chemical sensor systems without having to change the reporter gene (vide infra).

In the present invention, the combination of the iNOS reporter system with high-throughput in vivo techniques such as two-hybrid systems will provide a powerful technique for uncovering bioactive small molecules. A major advantage of this system not realized in current techniques is the ability to help identify the molecule responsible for a biological response. As discussed above, this is accomplished by tethering the NO sensor to the library beads and taking advantage of the secreted nature of the gene expression product (NO). By proximity, the beads which had delivered the active molecule will be exposed to the highest concentration of NO and, as a result, become the most fluorescent. These beads will be easily distinguishable from the others in the assay.

The rapid identification of bioactive molecules from a large combinatorial library in a whole cell format is an area of research which will benefit from interdisciplinary approaches, such as research incorporating organic chemistry and molecular biology. More specifically, the NO chemical reporter system of the present invention is highly integratable with current solid phase combinatorial synthesis techniques. In combination with current two-hybrid systems and various receptors, the iNOS system is a powerful technique for identifying small molecules that influence signaling pathways. The development of iNOS as a reporter gene system, in tandem with chemical sensors of NO and combinatorial organic synthesis, may help to expedite the drug discovery process.

These and other aspects of the present invention will be further appreciated upon consideration of the following Examples, which are intended to illustrate certain particular embodiments of the invention but are not intended to limit its scope, as defined by the claims.

### EXAMPLES

### Example 1

Identifying Small Molecules that Activate Expression of Nitric Oxide Synthase

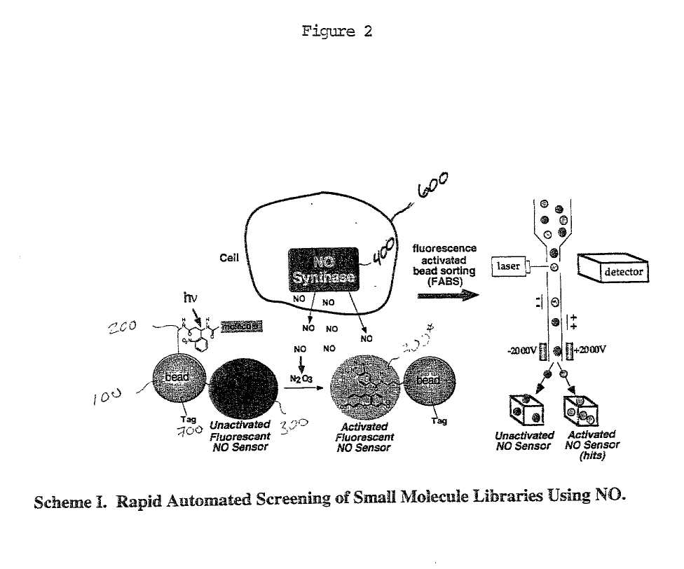

The present Example describes use of the inventive system to identify small molecules that activate expression of a nitric oxide (NO) reporter gene. The overall strategy is depicted in FIG. 2. As shown, an inducible NO synthase (iNOS) gene 400 is provided inside a cell 600. Expression of iNOS results in production of a protein product with the ability to generate large quantities of nitric oxide, a reactive gas which is rapidly secreted by activated cells (Hevel et al. J Biol. Chem. 266:22789, 1991; incorporated herein by reference).

Cells are contacted with solid phase resin beads 100 containing synthesized compounds 200 and a fluorescein-based sensor of nitric oxide (Nagano et al. FEBS 427:263, 1998; incorporated herein by reference). In the particular embodiment depicted in FIG. 2, the compounds 200 represent the products of a combinatorial synthesis, and the beads 100 also contain tags 700 recording the synthetic history of the beads according to known procedures (see, for example, Nestler et al. J. Org. Chem. 59:4723, 1994; incorporated herein by reference). Furthermore, the compounds 200 are attached to the beads 100 by means of a photocleavable linker and can be detached from the bead by photolysis. The released compounds can then exert their effects on the cell, e.g., by stimulating a signal transduction cascade or by entering the cell and affecting some intracellular process in a manner that results in expression of the iNOS reporter gene 400.

The NO synthase (NOS) protein encoded by the iNOS gene 400 catalyzes the conversion of arginine to nitric oxide and citrulline. iNOS remains active as long as arginine is present, thereby generating a large quantity of NO per binding event and effectively amplifying the signal initiated by a small molecule-protein interaction. NO is rapidly converted, under aerobic conditions. to N₂O₃ which will react with the NO sensor 300 on the bead converting it to a fluorescent bead (green->yellow; FIG. 2). The signal is amplified further because the activated fluorescein molecules are concentrated on the 130 micron beads rather than floating dilute in solution.

One advantage of the NOS-based reporter gene system, unlike other reporters such as luciferase, β-galactosidase, and SEAP is that there is no need to add additional developing agents following induction of gene expression. The catalytic activity of NOS, combined with concentration of activated fluorescent molecules on a small bead surface, enhances the sensitivity of this screening system.

In a preferred embodiment of the invention, the assay system of FIG. 2 is used to screen a small molecule library in a 1536 or 6000 well format. Beads containing active compounds are preferably identified by fluorescence-activated bead sorting (FABS; Baumann et al. J. Biol. Chem. 271:16500, 1996; Gallop et al. Proc. Natl. Acad. Sci. USA 90:10700, 1993; each of which is incorporated herein by reference) (FIG. 2). FABS, based upon fluorescence cell sorting techniques, is capable of sorting 10,000 beads/sec, which will allow the analysis of 1,000,000 beads (molecules)/100 sec. The encoding tags on the identified beads are then decoded, and the structures of active compounds are determined from the decoded information.

FABS has been used to separate hits from combinatorial libraries in the past, however these assays have involved in vitro binding assays between on-bead molecules and isolated proteins including the SH2 domains of Grb2 and Syk (Baumann et al. J Biol. Chem. 271:16500, 1996; incorporated herein by reference). The inventive system depicted in FIG. 2 analyzes events inside intact cells. The inventive system therefore has the advantage that it allows automated cell sorting of beads that contain compounds active in a whole cell assay. The inventive NOS-based assay system, when combined with FABS, constitutes a powerful method for cell-based ultra high-throughput screens for rapid identification of natural product-like compounds that bind specific protein targets.

### Example 2

Identification of Small Molecules that Influence the p53 Cell Signaling Pathway

This Example describes application of the inventive system to the identification of small molecules that influence the p53 cell signaling pathway.

The inventive system depicted in FIG. 2 is employed with the embellishment that the iNOS gene has been placed under the control of p53. Techniques for linking the iNOS gene to p53 control elements are well known in the art (see, for example, Molecular Cloning: A Laboratory Manual, 2nd Ed., ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999); Transcription and Translation (B. D. Hames & S. J. Higgins eds. 1984); each of which is incorporated herein by reference). Chemical compounds that affect p53 signaling are identified as described above in Example 1.

### Example 3

Identification of Compounds that Disrupt Protein-Protein Interactions

The present Example describes the use of the inventive system to identify compounds that interfere with protein-protein interactions.

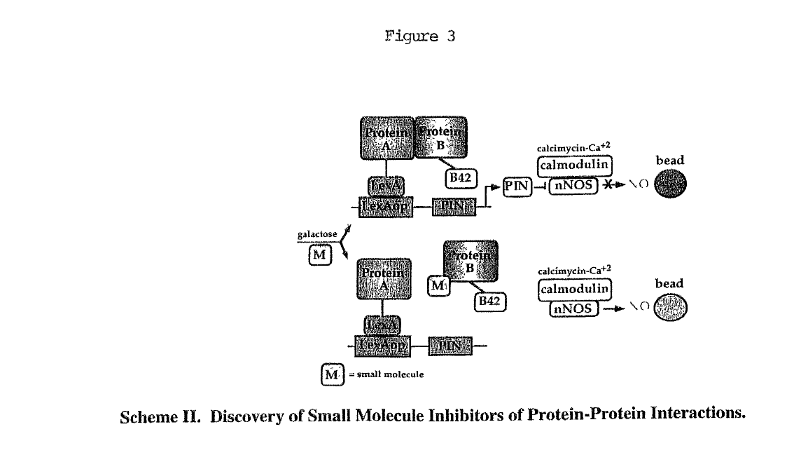

FIG. 3 depicts the use of the present inventive system to identify chemical compounds that disrupt a protein-protein interaction controlling expression of an NOS reporter gene. The so-called “two hybrid” transcriptional activation system is well established in the art. In general, a binding element for a DNA binding protein is positioned upstream of a gene. A hybrid protein including the DNA binding domain that recognizes the binding element and a first ligand domain is then provided. A second hybrid protein comprising a second ligand domain (that interacts with the first ligand domain) and a transcription activation domain is also provided. Interaction of the DNA binding domain with the binding element in the DNA recruits the first hybrid protein to the DNA; interaction between the first and second ligand domains then recruits the transcriptional activation domain to the DNA and activates transcription of the reporter gene. Any such two hybrid system may be used in the embodiment of the invention depicted in FIG. 3.

In the particular embodiment depicted in FIG. 3, a Lex A DNA binding domain is fused to a first regulatory ligand (Protein A in FIG. 3) and a second regulatory ligand (Protein B in FIG. 3) is fused to a B42 activation domain. To give but one example, protein A may comprise MDM2, and protein B may comprise p53.

The Lex A DNA binding site is then positioned upstream of a galactose-inducible promoter directing expression of a reporter gene (PIN in FIG. 3) encoding a recently-discovered protein (Jaffrey et al., Science 274:774, 1996; incorporated herein by reference) that inhibits the activity of nNOS, an isoform of NOS that requires calmodulin/Ca⁺² for activation.

A bead containing a small molecule and an NO sensor as described above in Example 1 is contacted with a system containing the reporter gene construct, the two interacting fusion A proteins, and nNOS. Preferably, the system comprises an intact cell in which each of the fusion proteins and the nNOS protein is expressed.

Preferably, the small molecule is then released from the bead and then galactose is added to the medium to allow expression of the PIN gene. It is generally preferred that the small molecule be released prior to addition of galactose so that the small molecule has the opportunity to act (e.g., to interfere with interaction of the first and second ligand domains) before reporter gene expression is monitored. Calcimycin is then added to the system, allowing nNOS to produce NO unless the PIN reporter gene has been activated. Thus, small molecules that disrupt the Protein A-Protein B interaction are identified because PIN expression is blocked in reactions containing those molecules, and therefore nNOS is activated in those reactions. nNOS activity results in NO production, which is detected by the NO sensor attached to the bead. Preferably, beads with activated NO sensors are identified by FABS, as described above in Example 1.

### Example 4

Detecting Small-Molecule-Induced Expression of the Nitric Oxide Synthase Gene

The present Example describes a system that we have developed and used to demonstrate that a solid-phase-bound sensor can detect expression of an NO-based reporter system.

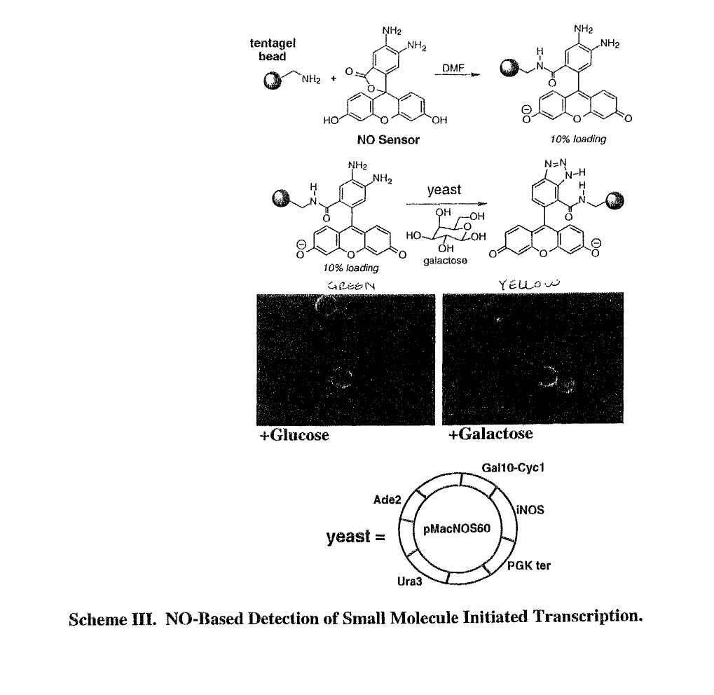

As shown in FIG. 4, we have synthesized a fluorescein-based NO sensor as described by Nagano et al. (FEBS 427:263, 1998, incorporated herein by reference) and have loaded it onto an animomethyl-TENTAGEL resin (130 μm) by mixing in DMF. The lactone function of the sensor is effectively aminolysed at room temperature over 48 hours. The apparent loading achieved was about 10% (0.03 mmol/g), although it is possible that much lower loading was in fact achieved.

We have also obtained a yeast strain developed by Mansuy et al (Sari et al. Biochemistry 35(22):7204-7213, June 1996; incorporated herein by reference) in which transcription of the NOS gene is under the control of the Gal10 promoter and is therefore inducible (and is referred to as iNOS). We grew the yeast cells in glucose media (SGI medium containing 7 g/L yeast nitrogen base without amino acids, 1 g/L casamino acids, 20 g/L glucose, and 50 mg/L tryptophan, at 30 ° C.) so that expression of the iNOS gene was repressed. The cells were harvested and washed with medium lacking any sugar (7 g/L yeast nitrogen base without amino acids, 1 g/L casamino acids and 50 mg/L tryptophan), were resuspended in the same sugar-free medium, and were distributed into 96-well plates.

The TENTAGEL beads containing sensor were washed with CH₂Cl₂, THF, DMF, i-PrOH, MeOH, EtOH, and deionized water, and were dried. 10 μl of sensor beads in sugar-free medium were added to wells containing 170 μl of yeast culture (20-30 beads per well). 20 μl of galactose solution was then added to each well, to give a final concentration of 100 mM (the final volume in each well was 200 μl. Galactose acts as a small-molecule inducer of iNOS expression, and contacted the cells with the beads containing the NO sensor. Control beads were treated with the same amount of galactose in the absence of yeast, or were incubated with cells along (in the absence of galactose). The beads were then washed with 200 μl MeOH and 200 μl deionized water twice, and were allowed to dry. A fluorescent microscope equipped with a CCD camera was used to visualize the beads.

As indicated in FIG. 4, a distinct color change (green ->yellow) was observed for those beads that were exposed to galactose-induced cells. A similar color change was observed for beads that were exposed to NO gas. Subsequent experiments have revealed that similar color changes may be observable with TENTAGEL itself, independent of sensor loading. As discussed below, strategies for improving sensor loading, and therefore for improving readout since presumably the NO sensor, even if not absolutely required, is a more effective indicator of NO than TENTAGEL alone, are being actively pursued.

This experiment demonstrates the feasibility of using an inventive NO-based fluorescent reporter/sensor system to detect a change in gene expression, and in particular to detect a gene expression change that is induced by a small molecule.

The beads used in this experiment are being sorted by FABS as described above in Example 1. Also, different reporter systems are being developed, in which the NOS coding sequence (or the PIN coding sequence) are placed under the control of different promoters and/or regulatory elements.

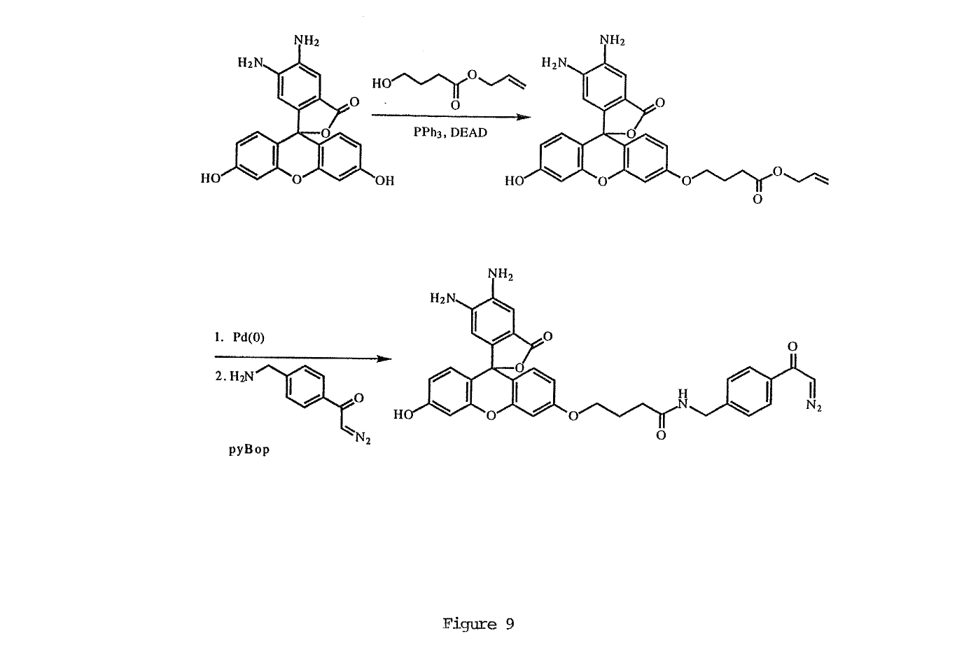

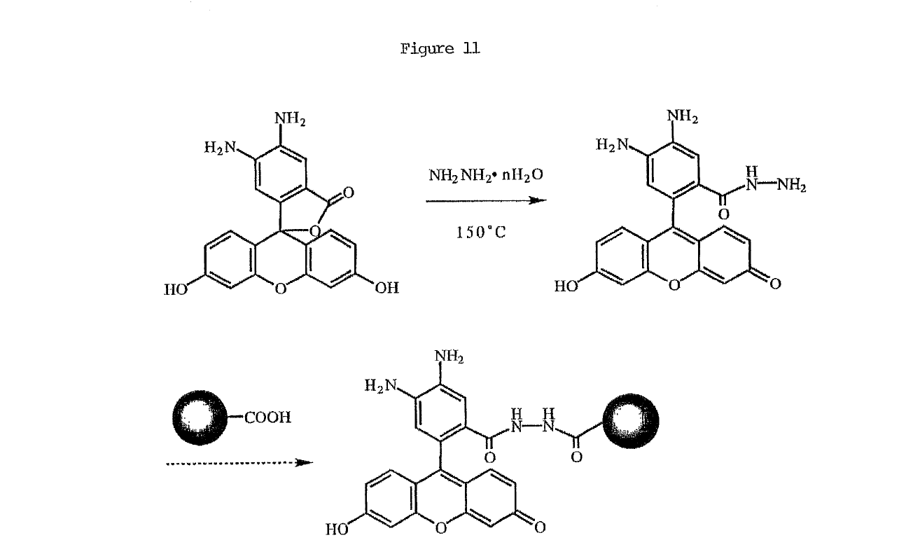

Alternative systems for coupling the sensor to the beads are under development. For example, one system, depicted in FIG. 9, utilizes carbene insertion. Another system, depicted in FIG. 11, utilizes hydrazine to create a chemical handle (a hydrazide) that allows reliable loading of sensor onto the resin via a standard amine coupling reaction. The only complication with this strategy is that we have found that, when treated with benzoic acid, both the hydrazide and the hydroxy group react. Since a free hydroxyl group may be required for fluorescence of the sensor (see Kojima et al. Chem. Pharm. Bull. 46:373, 1998; incorporated herein by reference), this strategy may not be desirable.

Other strategies to improve readout sensitivity include using a more powerful promoter than Gal10 and using multiple copies of the iNOS gene.

### Example 5

High-Throughput Screen for Small Molecules that Disrupt the Rb-E2F/DP1 Complex

This Example describes an inventive high throughput screen using a resin-bound NO sensor and an NOS reporter gene to identify small molecules that interfere with the interaction between Rb-E2F and DP1.

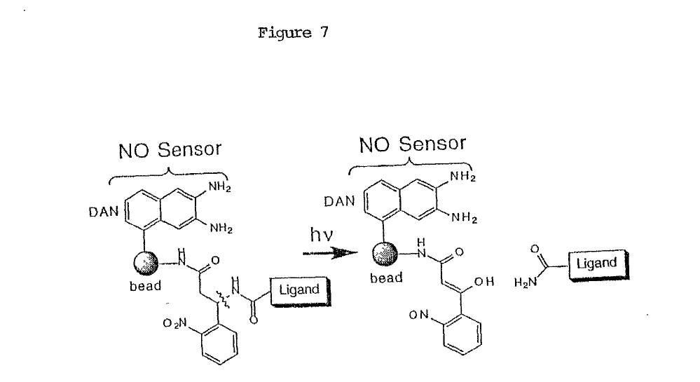

The NOS coding sequence is placed under the control of a galactose-inducible promoter to create an inducible NOS gene (iNOS). and regulatory element(s) is (are) added so that iNOS expression requires binding by the E2F transcription factor. DP1, which binds to E2F and is required for high affinity binding of E2F for Rb, is also provided. Rb represses the E2F-DP1 heterodimer and prevents transcription. The system is then contacted with beads containing small molecules and an NO sensor (eg., DAN molecules [see FIG. 7], preferably having been attached to the beads by olefin metathesis). Each bead contains approximately 50 pmoles of small molecule test compound, preferably attached to the bead by means of a photocleavable linker so that test compound is released from the bead upon mild photolysis.

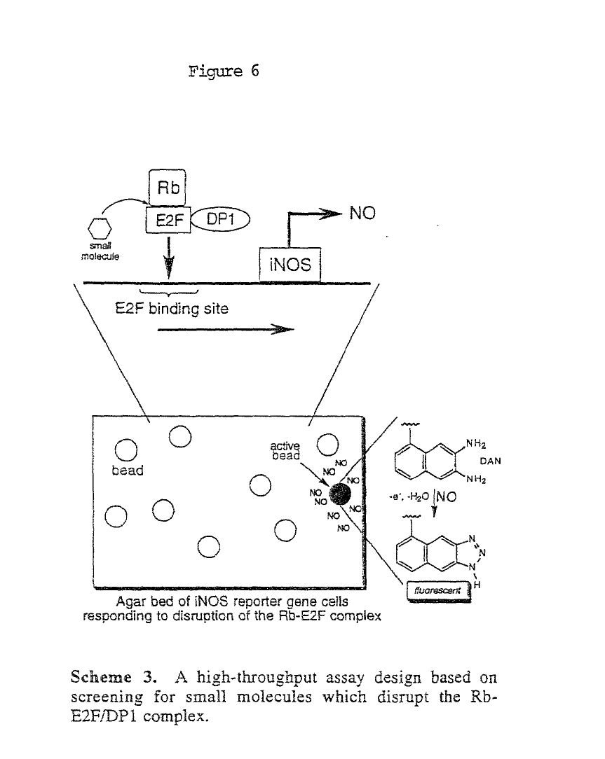

Approximately 10,000 beads are sprinkled on a bed of agar that also contains cells expressing the reporter construct and proteins described above. Test compounds are released from the beads and allowed to enter the cells. If a particular test compound is able to disrupt the Rb-E2F/DP1 interaction, NOS will be produced and NO gas will be secreted from cells that the test compound entered, which cells are in the physical vicinity of the bead from which the test compound was released (see FIG. 6). The DAN molecule attached to that bead will react with the NO and will undergo a nitrosation/cyclization reaction to generate a highly fluorescent product.

Because NO is rapidly oxidized to nitrite under aerobic conditions, and DAN does not react with nitrite at physiological pH, it is unlikely that NO will be present at significant, distances from the relevant bead (i. e., the bead that released the actual active compound). False positives are therefore minimized. The inventive system therefore provides a mechanism by which source beads containing active molecules can be individually identified, and preferably automatically sorted. No other system available allows this sort of designation of the particular bead, within a collection of beads, from which an active compound was released. Beads containing active compounds can then rapidly be identified, and the chemical structure of the active compound can be rapidly determined, for example by decoding synthetic history information stored in tags on the bead. Large amounts of the active compound can then be synthesized by simply repeating the steps encoded on the bead; derivatives of the active molecule can also be synthesized, for example by combinatorial chemistry, which would produce a new library of compounds, all related to the original active compound, and can then be screened as described herein.

### Example 6

### Assaying iNOS Expression in Mammalian Cells

This Example describes the use of a resin-attached NO sensor to assay NOS expression in mammalian cells in accordance with the present invention.

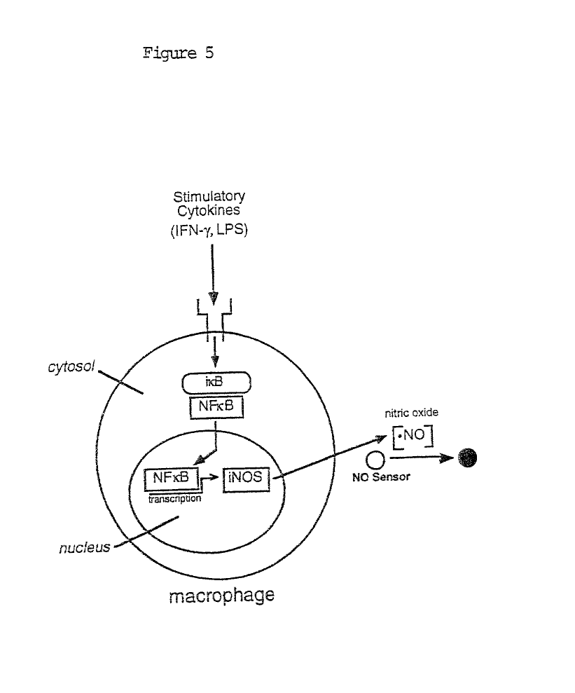

Macrophage cells are utilized that contain an iNOS gene under the tight control of the transcription factor NF-κB. In the basal state, NFκB is held in the cell cytoplasm through interaction with IκB, and the iNOS gene in the nucleus is dormant. Any of a wide variety of signals, including for example, extracellular TNFα, lipopolysaccharide (LPS), etc. can trigger NF-κB translocation to the nucleus and subsequent gene activation (Baltimore et al Curr. Opin, Biol. 5:477, 1993; incorporated herein by reference) (see FIG. 5).

The cells are contacted with solid phase resin containing releasable test compounds and an NO sensor. Test compounds are released from the resin, and those compounds that trigger NFκB translocation are identified because the iNOS gene is activated in the cells that they affect so that the cells are induced to produce NO and the NO sensors on the resins from which the compounds were released, which resins are necessarily in the physical vicinity of the cells, react to generate a readout (for example, calorimetric or fluorescent) which can be altered based on the assay and the desired response.

This system can be used to screen libraries of small molecules for their ability to activate the NFκB pathway. Such compounds have many uses, included uses as molecular probes to help elucidate the complex signaling pathway surrounding NFκB.

### Example 7

### Detection of NO from a Chemical Source Using a Resin-Bound NO Sensor

This Example describes use of an inventive solid-support-loaded NO sensor to detect NO from a chemical source.

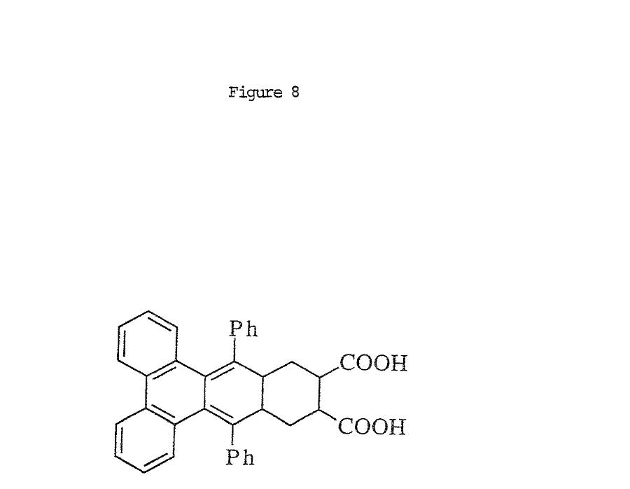

The NO sensor depicted in FIG. 8 was loaded on aminomethylated TENTAGEL beads (130 μm, 0.3 mmol/g) with an adjusted loading of 0.09 mmol/g. 20 mg of beads containing sensor were mixed with 10 ml sperinine NONOate solution (1 mM solution in pH 7.4 phosphate buffer) for 4 h. The beads were then mixed with 10 ml ascorbic acid solution (10 mM solution in pH 7.4 phosphate buffer) for another 3 h. After washing with deionized water and methanol and air dried, the beads were visualized using a fluorescent microscope equipped with a CCD camera.

### Example 8

### Detection of NOS Induction in Murine Macrophage Cells Using a Resin-Bound NO Sensor

This Example describes use of an inventive solid-support-loaded NO sensor to detect NO produced by induction of the NOS gene in a cell.

Murine macrophages RAW 264.7 were cultured at 37° C. with 5% CO₂ in DMEM containing 10% fetal bovine serum, 100 units/ml penicillin, and 10 μg/ml streptomycin. The cells were seeded into 96-well plates and incubated at 37° C. with 5% CO₂ for 1 day until the cells were grown to confluence. Medium was then removed, and the cells were washed with serum-free DMEM containing 1% Nutridoma SP, 100 units/ml penicillin, and 100 μg/ml streptomycin. 150 μl fresh serum-free medium was added to the wells and the cells were incubated for another day before the assay.

The TENTAGEL beads containing NO sensor prepared as described above in Example 7 (0.09 mmol/g) were washed with CH₂Cl₂, THF, DMF, i-PrOH, MeOH, EtOH, and deionized water, vacuum dried, and irradiated with a longwave UV lamp at 365 nm (Blak Ray, Model B 100 AP) for 10 min before the experiment. 10 μl of these beads in serum-free DMEM were added to each well (20-30 beads per well). To activate the macrophage, murine interferonγ (IFN-γ) and lipopolysaccharide (LPS) from Salmonella Abortus equi was added to give a final concentration of 10 units/ml and 10 ng/ml, respectively. The final volume in each well is 200 μl. As controls, the beads were treated with the same amount of IFN-γ and LPS in the absence of macrophage and also incubated with the cells alone. After 12 h of incubation (37° C., 5% CO₂), the medium was removed and 200 μl of methanol was added. The detached cells were removed. The beads were then washed with 200 μl methanol in the wells and allowed to dry. A fluorescent microscope equipped with a CCD camera was added to visualize the beads.

### Example 9

### Improved Linker System for Releasably Attaching Test Compounds to Solid Supports

The present Example describes an improved linker system that can be used to releasably attach test compounds to a solid support in a manner that will not interfere with readout of fluorescent signals from the support after the test compounds have been released.

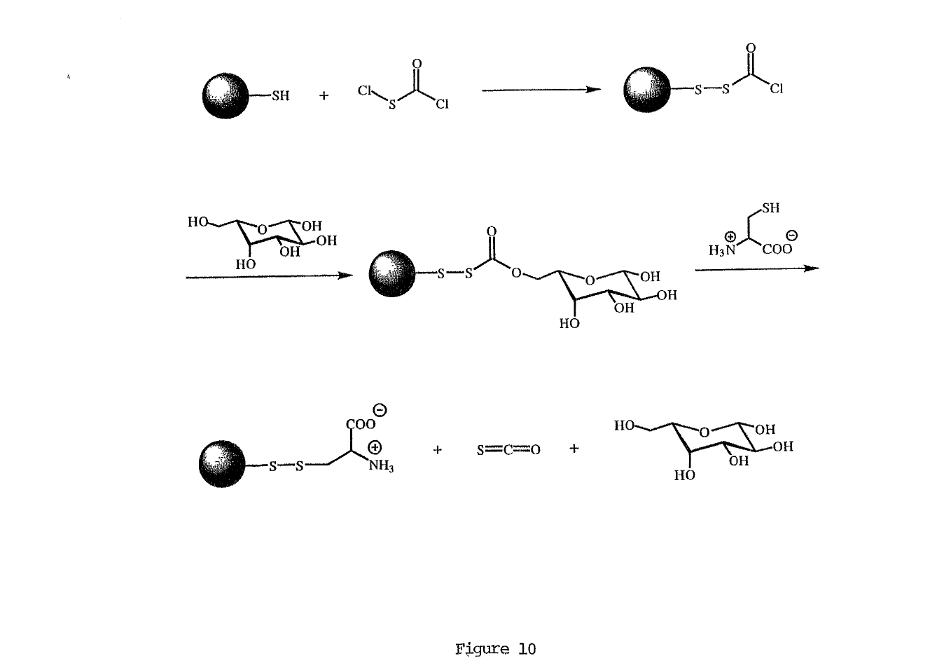

In the experiment described in Example 4, we found that some beads sometimes turned yellow even in the absence of galactose. This reaction appeared to be attributable to the particular photolinker employed in that experiment. Accordingly, we have developed an alternative linker system (see FIG. 10) that allows compounds to be released by addition of cysteine (see Smith et al., Biochemistry 14:766, 1975; incorporated herein by reference) rather than by photorelease. As this linker does not utilize a fluorophore, it should not affect the fluorescent properties of the solid support to which it is attached.

### Other Embodiments

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

We claim:

1. A method of identifying small molecules that affect a biological event of interest, the method comprising the steps of:
providing a collection of small molecules, each of which is attached to a solid support, which solid support is associated with a molecular sensor characterized in that at least one spectroscopic property of the sensor is altered in the presence of nitric oxide;
providing cells containing a reporter gene that encodes a nitric oxide synthase, expression of the reporter gene being indicative of occurrence or non-occurrence of a selected biological event involving the cells;
contacting the cells with the collection of small molecules; and
identifying solid supports with chemical sensors whose at least one spectroscopic property has become altered, the alteration in spectroscopic property revealing nitric oxide production, the production of nitric oxide revealing expression of the reporter gene, the reporter gene expression revealing occurrence or non-occurrence of the selected biological event, so that compounds attached or were attached to the identified solid support are revealed to have affected the biological event.

2. A method of identifying a test compound that affects a biological event of interest, the method comprising steps of:
providing a plurality of test compounds;
providing cells containing an inducible reporter gene, wherein expression of the reporter gene results in the production of a reporter gene product,
wherein the product is secreted by the cell,
wherein the product is detectable, and
wherein the presence of the products indicates occurrence or non-occurrence of a selected biological event;
contacting the cells with the plurality of test compounds; and
identifying test compounds which promote or inhibit a biological event based on production of the reporter gene product.

3. The method of claim 2, wherein the plurality of test compounds are attached to a solid support through a cleavable linkage.

4. The method of claim 2, wherein the linkage is severable by irradiation with light.

5. The method of claim 3, wherein the solid support is associated with a molecular sensor.

6. The method of claim 5, wherein the solid support is associated with a molecular sensor that can detect nitric oxide.

7. The method of claim 5, wherein the molecular sensor is 2,3-diaminonapthalene (DAN).

8. The method of claim 5, wherein the molecular sensor is diaminofluorescein.

9. The method of claim 4, wherein the molecular sensor is characterized in that at least one optical property of the sensor is altered in the presence of nitric oxide.

10. The method of claim 2, wherein the test compounds are small molecules.

11. The method of claim 2, wherein the plurality of test compounds is a combinatorial library of chemical compounds.

12. The method of claim 2, wherein the plurality of test compounds is a combinatorial library of small molecules.

13. The method of claim 2, wherein the test compounds are proteins.

14. The method of claim 2, wherein the test compounds are peptides.

15. The method of claim 2, wherein the test compounds are nucleic acids.

16. The method of claim 2, wherein the reporter gene encodes a reporter gene product that catalyzes the production of a chemical compound that is secreted by the cell.

17. The method of claim 2, wherein the reporter gene encodes a reporter gene product that is a small molecule.

18. The method of claim 2, wherein the reporter gene encodes a reporter gene product that catalyzes the production of a membrane permeable chemical compound that is detectable.

19. The method of claim 18, wherein the chemical compound is a gas at room temperature and 1 atm of pressure.

20. The method of claim 19, wherein the chemical compound is nitric oxide.

21. The method of claim 19, wherein the chemical compound is molecular oxygen.

22. The method of claim 19, wherein the chemical compound is carbon monoxide.

23. The method of claim 19, wherein the chemical compound is molecular nitrogen.

24. The method of claim 19, wherein the chemical compound is carbon dioxide.

25. The method of claim 2, wherein the cells are macrophages.

26. The method of claim 2, wherein the cells are yeast.

27. The method of claim 2, wherein the cells are mammalian cells.

28. The method of claim 2, wherein the cells are human cells.

29. The method of claim 2, wherein the cells are bacterial cells.

30. The method of claim 2, wherein the reporter gene is nitric oxide synthase.

31. The method of claim 2, wherein the reporter gene product is nitric oxide.

32. The method of claim 3, wherein the step of identifying comprises sorting the solid supports using fluorescence-activated bead sorting (FABS).

33. The method of claim 3, wherein the step of identifying comprises decoding tags on the solid support which correspond to the synthetic history of the test compound attached or was once attached to the bead or structural features of the test compound.

34. A library of test compounds attached to a solid support, wherein a chemical sensor is attached to the solid support.

35. The library of claim 34, wherein the chemical sensor is characterized in that at least one optical property of the sensor is altered in the presence of nitric oxide.

36. A method of synthesizing a combinatorial library, the method comprising steps of:
synthesizing a library of small molecules on a solid support by combinatorial chemistry methods;
attaching tags to the solid support which encode the synthesis or structural element; and
attaching a molecular sensor to the solid support able to detect the reporter gene product.

37. A method of providing a molecular sensor, the method comprising steps of:
providing a collection of chemical compounds linked to a solid support; and
attaching a molecular sensor to the solid support.

38. An assay system comprising a nitric oxide synthase gene, wherein the gene is a reporter gene.

39. An assay system comprising a nitric oxide synthase gene, wherein the gene is a reporter gene, and a molecular sensor capable of detecting nitric oxide.

40. An assay system comprising
a first construct encoding a DNA binding domain fused to a first protein of interest;
a second construct encoding an activation domain fused to a second protein of interest; and
a third construct comprising a binding site of the DNA binding domain functionally lined to a reporter gene.

41. The assay system of claim 40, wherein the DNA binding domain is LexA.

42. The assay system of claim 40, wherein the activation domain is B42.

43. The assay system of claim 40, wherein the reporter gene is nitric oxide synthase.

44. The assay system of claim 40, wherein the reporter gene is protein inhibitor of nitric oxide synthase (PIN).

45. The assay system of claim 40, wherein the first or second protein of interest is MDM2.

46. The assay system of claim 40, wherein the first or second protein of interest is p53.

47. A method of synthesizing and assaying a library of small molecules. the method comprising steps of:
synthesizing a library of small molecules on a solid support by combinatorial chemistry;
encoding a synthesis with an encoding tag attached to the solid support;
attaching a molecular sensor to the solid support wherein at least one spectroscopic property of said sensor is altered by chemical reactions involving nitric oxide;
providing cells containing a DNA sequence encoding a nitric oxide synthase wherein a protein-protein interaction of interest affects transcriptional activation of said sequence;
contacting cells with the solid supports containing the library, the encoding tag, and the sensor, wherein each cell is exposed to no more than one solid support;
releasing the small molecules from the solid supports;
contacting the cell with the small molecule;
allowing the small molecule to affect the protein-protein interaction;
allowing transcriptional activation of the DNA encoding a nitric oxide synthase;
allowing production of nitric oxide by a nitric oxide synthase;
allowing chemical reactions involving nitric oxide to alter at least one spectroscopic property of the sensor;
identifying solid supports with sensors having an altered spectroscopic property; and
decoding the encoding tag of sensors having an altered spectroscopic property to identify the small molecule affecting a protein-protein interaction of interest.
